(19) United States
(12) Patent Application Publication
(10) Pub. No.: US 2004/0117875 A1
(43) Pub. Date: Jun. 17, 2004

(54) Systems and methods for delivering interferon to a subject

(76) Inventors: Amit Gal-On, Ramat Hasharon (IL); Yoel Shiboleth, Kibbutz Magal (IL); Tsachi Arazi, St. Kiryat Ono (IL); Yaron Ilan, Jerusalem (IL)

Correspondence Address: Mark M Friedman, Bill Polkinghorn, Discover Dispatch, 9003 Florin Way, Upper Marlboro, MD, 20772, US

(21) Appl. No.: 10/432,777

(22) PCT Filed: Nov. 28, 2001
(86) PCT No.: PCT/IL01/01099

(51) Int. Cl. C12Q 1/68; A61K 48/00; A01H 1/00; C12N 15/82

(52) U.S. Cl. 800/288; 424/93.21

(57) ABSTRACT

Systems and methods for providing supplemental interferon to a subject. One disclosed system includes a viral vector capable of infecting a plant and expressing interferon therein and the plant, which is edible. Another disclosed system includes a DNA capable of expressing an interferon gene in a plant and the plant, which is edible and susceptible to transformation by the DNA sequence. Further disclosed is a method including causing a plant to express at least a portion of an interferon gene and feeding at least a portion of the plant to the subject.

### FIELD AND BACKGROUND OF THE INVENTION

The present invention relates to systems and methods for providing supplemental interferon to a subject and, more particularly, to systems and methods of administering interferon via an edible plant. The present invention further relates to a general method for providing an orally bio-available protein to a subject.

In the last decade the use of plant viruses as vectors for gene expression of numerous proteins has received considerable attention, and several RNA virus vectors have been developed (Takamatsu et al., 1987; Chapman et al., 1992; Dolja et al., 1992; Kumagai et al., 1993; Rommens et al., 1995; Porta and Lomonossoff, 1996; Scholthof et al., 1996; Arazi et al., 2001). These vectors have been successfully used for in planta expression of plant genes (Hammond-Kosack et al., 1995; Sablowski et al., 1995; Kumagai et al., 2000) and heterologous genes (Hamamoto et al., 1993; Hendy et al., 1999; McCormick et al., 1999; Gopinath et al., 2000; Zhang et al., 2000). Unfortunately, since most known plant viruses cause significant yield losses to host plants use of these plant virus vectors for the production of commercial crops with improved agronomic traits, or with added nutritional or pharmaceutical value has not been feasible.

In addition, viruses are transmitted to other plants by their natural vectors in the field (Matthews, 1991). This issue raises serious concerns for use of plant virus vectors in the field.

Zucchini yellow mosaic virus (ZYMV) is one of the most devastating diseases worldwide of cucurbit species such as cucumber, squash, melon and watermelon (Desbiez and Lecoq, 1997). ZYMV is a member of the potyviridae family, the largest group of plant-infecting viruses (Shukla et al., 1994). As in all potyviruses, the ZYMV genome consists of a single messenger-polarity RNA molecule of about 10 kb, encapsidated by multiple copies of a single coat protein (CP) forming a flexuous filamentous particle (Gal-On et al., 1992 J. Gen. Virol. 73: 2183-2187.). Viral RNA is translated into a large polyprotein that is proteolytically processed to 8-9 functional proteins by three virus-encoded proteases: P1, HC-Pro and NIa (Riechmann et al., 1992, Revers et al., 1999). The P1 (Verchot et al., 1991) and HC-Pro (Carrington et al., 1989) proteinases are the first and second proteins located at the N′-terminus region of the polyprotein and catalyze autoproteolytic cleavage at their own C′-terminus. The NIa protease is responsible for cis and trans proteolytic cleavages of the remainder of the viral polyprotein (Carrington et al., 1988; Riechmann et al., 1992).

Theoretically, potyviruses are promising expression vectors, since their proteolytic processing strategy of gene expression requires that a foreign protein, synthesized as part of the viral polyprotein, is produced in equimolar amounts with all viral proteins (Riechmann et al., 1992; Revers et al., 1999). Moreover, taking into account the helicoidal morphology of viral particles, no packaging limitations would be expected for rather large genome insertions (Dolja et al., 1992; Scholthof et al., 1996). Expression of foreign genes by potyviruses has been demonstrated in tobacco etch virus (TEV) (Dolja et al., 1992), plum pox virus (PPV) (Guo et al., 1998), lettuce mosaic virus (LMV) (Choi et al., 2000; German-Retana et al., 2000). In these studies, foreign genes were inserted between the P1 and the HC-Pro genes, and were expressed as an insertional fusion with the N-terminus of the HC-Pro gene. Alternatively, a non-fused foreign gene expression was established by addition of the appropriate proteolytic cleavage sites to the ends of the foreign gene sequence (Dolja et al., 1997; Guo et al., 1998; Choi et al., 2000; Masuta et al., 2000). However, in these prior art studies, utility was limited by genetic instability of the constructs due to RNA recombination events that rapidly eliminated foreign sequences (Dolja et al., 1993; Guo et al., 1998; Choi et al., 2000). This appeared to be a serious inherent limitation of the system.

More recently, Masuta et al., demonstrated that a foreign gene expressed via clover yellow vein virus vector in legumes was genetically stable (Masuta et al., 2000). Howa-ever, as Masuta admits, “ . . . the present form of the CIYVV vector is that it retains its ability to induce lethal necrosis of host plants”. This disadvantage effectively renders the teachings of Masuta useless for commercial production of protein in edible legume crops. This problem is typical of prior alt potyvirus vectors created to date.

Interferon holds considerable promise as a drug in treating a number of medical conditions because of its therapeutic capabilities. Interferon is a naturally occurring protein with immuno-modulatory and anti-viral properties, that is produced in cultured human cells or in E. coli as a drug (reviewed by Walter et al., 1998). Interferon-alpha and Interferon-beta are both Type I interferons. Type I interferons are a large class of naturally-occurring cytokines which includes over 16 subclasses of IFN-alpha, plus IFN-beta and IFN-omega. The Type I interferons bind to a single cell surface receptor, and stimulate a complex sequence of signal transduction events leading ultimately to anti-viral, anti-proliferative and other immunomodulatory effects, cytokine induction, and HLA class I and class II regulation (Pestka et al., Annu. Rev. Biochem., 1987 56: 727). Alpha interferons are used widely for the treatment of a variety of haematological malignancies including hairy cell leukaemia, chronic myelogenous leukaemia, low grade lymphomas, cutaneous T-cell lymphomas, and solid tumours such as renal cell carcinoma, melanoma, carcinoid tumours and AIDS-related Kaposi's sarcoma (Gutterman, J. U., Proc. Natl. Acad. Sci. USA, 1994 91: 1198-1205). Anti-tumor effects are usually seen at high dosage levels, often of the order of tens of millions of units of interferon-alpha, administered by parenteral injection. Interferon-beta is licensed for clinical use in treatment of relapsing-remitting multiple sclerosis and chronic viral hepatitis B and C.

A commercial interferon alpha 2a (Roferon-A; see http://www.rocheusa.com/products/roferon) is claimed to normalize serum ALT, improve liver histology and reduce viral load in patients with chronic hepatitis C. The product is further indicated for the treatment of chronic hepatitis C, hairy cell leukemia and AIDS-related Kaposi's sarcoma in patients 18 years of age or older. In addition, it is indicated for chronic phase, Philadelphia chromosome positive chronic myelogenous leukemia (CML) patients who are minimally pretreated (within 1 year of diagnosis). While the manufacturer's claims may serve to establish the need for interferon alpha, they do not provide a means for producing interferon, nor a means of safely delivering IFN without the expense and complication of purifying the drug.

Although a number of routes of administration, including intravenous, subcutaneous, intramuscular, topical, and intralesional injection, are commonly employed for the administration of type I interferons, the oral route has not been generally used, because interferons are proteins which ate considered to be inactivated by proteolytic enzymes.

It is widely considered that in order to obtain the maximum therapeutic effect, the highest possible dose of interferon should be used. Although the availability of recombinant material has meant that very high dose levels are feasible, in practice it has been found that the side-effects of interferon administration have severely limited the dose of interferon which can be used and the duration of treatment. These side-effects include severe malaise and depression, leading in some cases even to suicide. A recent editorial by Hoofnagle in the New England Journal of Medicine has summarized these problems (Hoofnagle, J. H., and Lau, D., New Eng. J. Medicine 1996, 334: 1470-1471). Meta-analysis of the effect of interferon-alpha treatment in patients with chronic hepatitis B has shown a rate of remission of 25 to 40%, in patients with typical chronic hepatitis B, treated with 5 million international units (IU) daily or 10 million IU three times per week for 3 to 6 months. These results fall short of a cure, however, as most patients remain positive for hepatitis surface antigen and harbor viral DNA when tested by the polymerase chain reaction. Furthermore, these doses of interferon are poorly tolerated, and 10% to 40% of patients require dose reduction due to intolerable side effects. At a well-tolerated dose of 1 million IU daily, the remission rate is, however, only 17% (Perrillo et al. News Eng. J. Medicine, 1990, 323: 295-301). In patients with chronic hepatitis C, sustained long-term improvement is associated with the loss of HCV RNA, which occurs in only 10 to 20% of patients treated with a dose of 3 million IU three times per week for 6 months (Hoofnagle and Lau, op. cit.). In patients with cancer, significant response rates are usually seen only at the highest tolerated doses of interferon-alpha. Thus in patients with multiple myeloma, for example, the response rate is 50% in patients treated with 20 to 30 million IU daily, and only 15 to 20% in patients treated with 3 million IU. Very few patients are able, however, to tolerate the high-dose regimen for more than a short period of time (Ahre et al. Eur. J. Hematol., 1988, 41: 123-130). Thus clearly there is a need in the art for means, which would enable the administration of high dose interferon without the induction of severe side-effects.

There have been a number of anecdotal reports of efficacy of low doses of interferon administered as a nasal spray or as an oral liquid formulation in the treatment of a variety of viral conditions, particularly influenza. Placebo-controlled trials of relatively high dose intranasal interferon for treatment of rhinovirus infection showed that the treatment was effective, but that there was a significant incidence of side-effects (Hayden et al, J: Infect. Dis., 1983 148: 914-921; Douglas et al, New Engl. J. Med., 1986 314: 65-80; Hayden et al, New Engl. J. Med., 1986 314: 71-75).

More recently a series of patent specifications has described the use of low doses of orally administered interferon of heterologous species origin for the treatment of infectious rhinotracheitis (“shipping fever”) in cattle, and of feline leukaemia, and also treatment of other conditions, for enhancement of efficiency of vaccines; for improving the efficiency of food utilisation; and for prevention of bovine theileriosis. See U.S. Pat. No. 4,462,985, Australian Patent No. 608519, Australian Patent No. 583332 and U.S. Pat. No. 5,215,741 respectively. In addition U.S. Pat. No. 5,017,371 discloses the use of interferon in this way for treatment of side-effects of cancer chemotherapy or radiotherapy. In these specifications, the interferon used was human interferon-alpha prepared by the method of Cantell, administered in phosphate buffered saline, at a dose of 0.01 to 5 IU per pound body weight. While these specifications suggest that such low doses of interferon administered to the oropharyngeal mucosa, preferably in a form adapted for prolonged contact with the oral mucosa, may be efficacious for treatment of a wide variety of conditions including cancer, the experimental evidence for conditions other than shipping fever, feline leukaemia, canine parvovirus and theileriosis is largely anecdotal. In particular, no properly controlled trials of this treatment in any animal model for human cancers are presented.

More recent studies on the effects of very low doses of interferon administered by the oral or oropharyngeal mucosa have been reviewed (Bocci, Clin. Pharmacokinet., 1991 21: 411-417; Critic. Rev. Therap. Drug Carrier Systems, 1992 9: 91-133; Cummins and Georgiades, Archivum Immun. Therap. Exp., 1993 41: 169-172). It has been proposed that this type of treatment is particularly useful for treatment of HIV infection, and can at least improve quality of life in AIDS patients (Kaiser et al, AIDS, 1992 6: 563-569; Koech et al, Mol. Biol. Ther., 1990 2: 91-95). However, other reports indicate that such treatments provide no clinical benefit. A Phase I study of use of oral lozenges containing low doses of interferon for treatment of hepatitis B has also been reported (Zielinska et al, Archiv. Immunol. Therap. Exp., 1993 41: 241-252).

U.S. Pat. No. 6,207,145 to Tovey teaches high dose oro-mucosal administration of interferon. Teaching of this patent do not include means of manufacturing the interferon, nor of purifying the interferon from, for example, a culture of E. coli.

A series of United States patent applications (U.S. Pat. Nos. 5,817,307; 5,824,300; 5,830,456; 5,846,526; 5,882,640; 5,910,304 and 6,036,949) deal with various uses of orally administered interferon. The teachings of U.S. Pat. No. 5,817,307 are limited to saliva soluble solid dosage forms of interferon. The teachings of U.S. Pat. Nos. 5,824,300; 5,830,456; 5,846,526 and 5,882,640 are similarly limited. This is because the prior art teaches that the environment in the mammalian digestive tract renders interferon inactive. Thus, these patents teach against delivery of interferon in the digestive tract, for example as a saliva insoluble plant cell containing interferon within the cellulose wall of a plant cell. The teachings of U.S. Pat. No. 5,910,304 require the administration of interferon in solution. The teachings of U.S. Pat. No. 6,036,949 require that the interferon be administered in a “pharmaceutically acceptable” solid or liquid form. Saliva solubility is again taught. None of these patents teach administration of interferon without the need for purifying the drug and “formulating” it in a controlled fashion. Therefore, all of these teachings require expensive industrial manufacturing processes, in stark contrast to the invention claimed herein.

Current production techniques are ill suited to meet the demand for interferon in treating these prevalent diseases. In addition, purification of interferon from cultured cells makes the cost of interferon treatment high. Further, much of the commercially available interferon currently available is in injectable form. U.S. Pat. No. 5,766,885 to Carrington et al. teaches potyvirus vectors for expression of foreign genes. Carrington specifically teaches “A method for expressing at least one protein in a plant or plant cell, said method comprising infecting a plant or plant cell susceptible to a polyprotein-producing potyvirus with said potyvirus, expressing said potyvirus to produce said polyprotein, wherein said potyvirus codes for at least one protein non-native to the potyvirus and wherein said non-native protein is released from said polyprotein by proteolytic processing.” However, these teachings contain neither a hint nor a suggestion that such a non-native protein would be orally bio-available.

Further, the teachings of Carrington include hypothetical production of insulin, hGH, interleukin, EPO, G-CSF, GM-CSF, hPG-CSF, M-CSF, Factor VIII, Factor IX, and tPA although no enabling support is provided in the specification thereof. Because of the potent biological activity of these compounds, it is not clear from the reporter gene examples used by Carrington whether production of pharmaceuticals in plants is feasible at all. Carrington himself (example 3) characterizes his claim for insulin production as “prophetic”. Such a teaching constitutes an admission by the inventor that the invention was not in hand at the time of filing.

There is currently much interest in developing additional uses for orally administered interferon (Bocci 1999; Cummins et al., 1999; Fleischmann et al., 1999; Ship et al., 1999 and Tompkins, 1999). This interest heightens the importance of the disclosed invention in providing a viable means for production and supply of orally bio-available interferon.

There is thus a widely recognized need for, and it would be highly advantageous to have, systems and methods for providing supplemental interferon, and other orally bio-available proteins, to a subject, devoid of the above limitations.

### SUMMARY OF THE INVENTION

According to one aspect of the present invention there is provided a system for providing supplemental interferon to a subject. The system includes: (a) a viral vector, the vector designed and constructed to be capable of infecting a plant and expressing at least a portion of an interferon gene therein and (b) the plant, at least a portion of the plant being edible by the subject. The gene product of the at least a portion of an interferon gene is bio-available to the subject consuming the at least a portion of said plant.

According to another aspect of the present invention there is provided a system for providing supplemental interferon to a subject. The system includes: (a) a DNA sequence designed and constructed to be capable of expressing at least a portion of an interferon gene in a plant; and (b) the plant, at least a portion of the plant being edible by the subject and the plant susceptible to transformation by the DNA sequence. The gene product of the at least a portion of an interferon gene is bioavailable to the subject consuming the at least a portion of said plant.

According to yet another aspect of the present invention there is provided a method for providing supplemental interferon to a subject. The method includes the steps of: (a) causing a plant to express at least a portion of an interferon gene in at least some cells thereof; and (b) feeding at least a portion of the plant to the subject.

According to still another aspect of the present invention there is provided a method for providing an orally bio-available protein to a subject.

The method includes the steps of: (a) causing a plant to express at least a portion of the orally bio-available protein in at least some cells thereof; and (b) feeding at least a portion of the plant to the subject.

According to further features in preferred embodiments of the invention described below, the viral vector is a potyvirus vector.

According to still further features in the described preferred embodiments the potyvirus is zucchini yellow mosaic virus (ZYMV).

According to still further features in the described preferred embodiments the ZYMV is an attenuated strain containing a mutation as listed in SEQ ID NOs.: 7 and 8.

According to still further features in the described preferred embodiments the at least a portion of an interferon gene includes a mammalian interferon gene sequence.

According to still further features in the described preferred embodiments the mammalian interferon gene sequence includes at least a portion of a human interferon gene sequence.

According to still further features in the described preferred embodiments the human interferon gene sequence is selected from the group consisting of interferon alpha 2a (SEQ ID NO.: 1)

and any gene at least 85% homologous thereto as analyzed by the FastA program. The FASTA program family (FastA, TFastA, FastX, TFastX and SSearch) was written by Professor William Pearson of the University of Virginia Department of Biochemistry (Pearson and Lipman, Proc. Natl. Acad. Sci., USA 85; 2444-2448 (1988)). In collaboration with Dr. Pearson, the programs were modified and documented for distribution with GCG Version 6.1 by Mary Schultz and Irv Edelman, and for Versions 8 through 10 by Sue Olson. As used herein “analyzed by the FastA program” indicates analysis using default parameters of the program as currently specified.

According to still further features in the described preferred embodiments the vector expresses at least a portion of a protein selected from the group consisting of the interferon alpha 2a gene product (SEQ ID NO.: 2) and any protein at least 85% homologous thereto as analyzed by the FastA program.

According to still further features in the described preferred embodiments transmissibility of the viral vector from the plant to a second plant is prevented by a mutation in the viral vector.

According to still further features in the described preferred embodiments the system further includes a means for introducing the DNA sequence into at least one cell of the plant, thereby transforming the cell.

According to still further features in the described preferred embodiments the DNA sequence includes a left border and a right border of the agrobacterium T-DNA.

According to still further features in the described preferred embodiments the step of causing is accomplished by an action selected from the group consisting of: (i) infecting at least one cell of the plant with a viral vector, the viral vector designed and constructed to be capable of expressing at least a portion of an interferon gene therein; and (ii) transforming at least one cell of the plant with a DNA sequence designed and constructed to be capable of expressing at least a portion of an interferon gene therein.

According to still further features in the described preferred embodiments the step of causing is accomplished by an action selected from the group consisting of: (i) infecting at least one cell of the plant with a viral vector, the viral vector designed and constructed to be capable of expressing at least a portion of a gene encoding the orally bio-available protein therein; and (ii) transforming at least one cell of the plant with a DNA sequence designed and constructed to be capable of expressing at least a portion of a gene encoding the orally bio-available protein therein.

According to still further features in the described preferred embodiments the human interferon gene sequence is selected from the group consisting of interferon beta (SEQ ID NO.: 11) of interferon gamma (SEQ ID NO.: 13) and any gene at least 85% homologous to either of the interferon genes as analyzed by the FastA program.

According to still further features in the described preferred embodiments the vector expresses at least a portion of a protein selected from the group consisting of the interferon beta gene product (SEQ ID NO.: 12), the interferon gamma gene product (SEQ ID NO.: 14) and any protein at least 85% homologous to either of the interferon gene products as analyzed by the FastA program.

The present invention successfully addresses the shortcomings of the presently known configurations by providing systems and methods of providing supplemental interferon, or other orally bio-available proteins, to a subject.

### BRIEF DESCRIPTION OF THE DRAWINGS

The invention is herein described, by way of example only, with reference to the accompanying drawings and photographs. With specific reference now to the figures in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and B depict a viral vector for use in conjunction with a system according to the present invention;

FIGS. 2A and B illustrate stability and accumulation of recombinant AGII in plants by means of an immunoblot and histogram;

FIGS. 3A-D illustrate that AGII-interferon alpha-2a (AGII-IFN) does not affect cucumber development or yield, and is stable in planta by means of photographs, histograms and an RT PCR analysis;

FIGS. 4A-C illustrate AGII-IFN-mediated synthesis of IFN in squash and cucumber leaves by means of histograms and an immunoblot;

FIGS. 5A-D illustrate AGII-IFN mediated synthesis of IFN in squash and cucumber fruits and fruit parts as histograms; and

FIGS. 6A-H illustrate expression of foreign proteins in various plant parts via AGII vector.

### DESCRIPTION OF THE PREFERRED EMBODIMENTS

The present invention is of systems and methods for providing supplemental interferon to a subject. Specifically, the present invention can be used to deliver interferon orally as a portion of an edible plant, for example a cucurbit fruit such as cucumber, squash or melon. The present invention further relates to a general method for providing an orally bio-available protein to a subject.

The principles and operation of systems and methods for providing supplemental interferon (and other orally bio-available proteins) according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

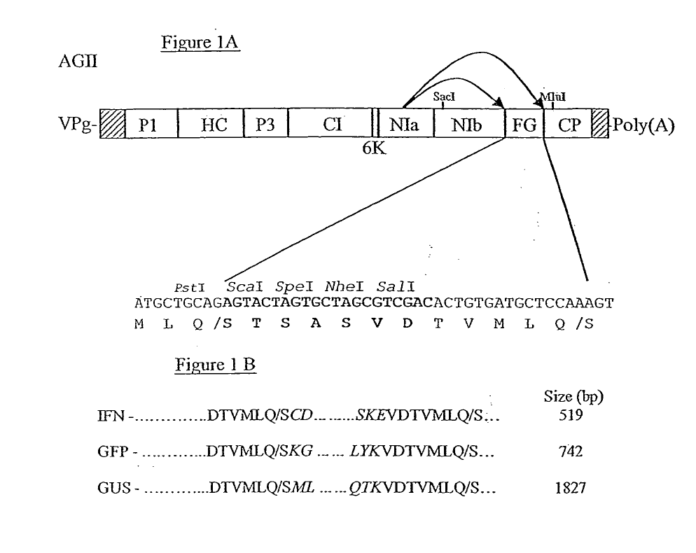

The present invention is embodied in part by a system for providing supplemental interferon to a subject. Referring now to the drawings, FIGS. 1A and B illustrate a viral vector for use as part of a system according to the present invention. Specifically, the AGII strain of ZYMV with IFN gene inserted into its genome is illustrated. FIG. 1A is a schematic presentation of the AGII genome. AGII non-coding (hatched shading), and coding (open boxes) regions including the inserted foreign gene (FG) are shown. Arrows indicate NIa protease involved in proteolysis of foreign gene product. NIa cleavage sites are indicated by /. Restriction enzyme sites used for sub-cloning are indicated. Nucleotides specifying restriction endonuclease recognition sites, inserted to create the polylinker and their encoded amino acid residues are indicated in bold in FIG. 1B. Insertion of interferon gene occurs between the NIb and CP genes. Amino acid sequence is indicated by italics.

The viral vector of the system is designed and constructed to be capable of infecting a plant, expressing at least a portion of an interferon gene therein. Therefore, the gene product of the at least a portion of an interferon gene is bio-available to the subject consuming the at least a portion of said plant. Delivery may be effected, for example, using what is commonly referred to as a “gene gun” by those ordinarily skilled in the art. Preferably, the viral vector is a potyvirus vector, more preferably the potyvirus is zucchini yellow mosaic virus (ZYMV), more preferably still the ZYMV is an attenuated strain, for example one containing a mutation as listed in SEQ ID NOs.: 7 and 8, the ZYMV-AGII engineered strain.

The at least a portion of an interferon gene may include a mammalian interferon gene sequence or a recombinant interferon gene derived from a combination of naturally occurring interferon genes. The mammalian interferon gene sequence may include, for example, at least a portion of a human interferon gene sequence including, but not limited to, interferon alpha 2a (SEQ ID NO.: 1). Alternately, or additionally, the mammalian interferon gene may include at least a portion of a gene at least 85% homologous to the interferon 2 alpha gene as analyzed by the FastA program.

Alternately, or additionally, the human interferon gene sequence may be an interferon beta, for example SEQ ID NO.: 11 or an interferon gamma, for example, SEQ ID NO.: 13 or any gene at least 85% homologous to either of these interferon genes as analyzed by the FastA program.

Alternately, or additionally, the vector may express at least a portion of an interferon beta gene product, for example, SEQ ID NO.: 12, or an interferon gamma gene product, for example, SEQ ID NO.: 14 or any protein at least 85% homologous to either of these interferon gene products as analyzed by the FastA program.

Once it has been delivered to the plant, the vector expresses at least a portion of a protein including, but not limited to, the interferon alpha 2a gene product (SEQ ID NO.: 2) or any protein at least 85% homologous thereto as analyzed by the FastA program. FastA may be implemented, for example, as part of the BLAST or GCG program packages. BLAST and FastA are services offered by the NCBI of the National library of Medicine of the National Institutes of Health. Both are accessible via the Internet, and one ordinarily skilled in the art of molecular biology will be familiar with access and use thereof.

Because of environmental concerns, it is preferable that transmissibility of the viral vector from the plant to a second plant is prevented by a mutation therein.

The system of the present invention further includes the plant, at least a portion of which is edible by the subject.

The present invention is further embodied by a system for providing supplemental interferon to a subject. The system includes a DNA sequence designed and constructed to be capable of expressing at least a portion of an interferon gene in a plant. The interferon gene is as described hereinabove. The system further includes the plant, at least a portion of which is edible by the subject. According to this system, the plant is susceptible to transformation by the DNA sequence. Preferably, the system further includes a means for introducing the DNA sequence into at least one cell of the plant, thereby transforming the cell. These means may include, for example, what is commonly referred to as permanent or transient “agrobacterium mediated transformation” or use of what is commonly referred to as a “gene gun” by those ordinarily skilled in the art of plant transformation.

Further, the DNA sequence itself may include portions designed to facilitate genetic transformation of plant cells. These portions may include, for example, a left border and a right border of the agrobacterium T plasmid.

The present invention is further embodied by a method for providing supplemental interferon to a subject. The method includes the step of causing a plant to express at least a portion of an interferon gene in at least some cells thereof. For purposes of this specification and the accompanying claims, the phrase “at least some cells thereof” refers to cells found within a plant, seeds thereof, and tissue culture cells derived therefrom. The method further includes the step of feeding at least a portion of the plant to the subject. The interferon is as described hereinabove. It will be appreciated that the step of causing may be accomplished in a wide variety of ways.

For example, “causing” may include infecting at least one cell of the plant with a viral vector. In this case, the viral vector is designed and constructed to be capable of expressing at least a portion of an interferon gene within the infected cell. Preferably the vector is further designed and constructed to cause assembly of virions, which infect adjacent cells. More preferably, delivery to a single cell of the plant results in systemic infection of the plant.

Alternately, “causing” may include transforming at least one cell of the plant with a DNA sequence designed and constructed to be capable of expressing at least a portion of an interferon gene therein. Such a transformation may be either a somatic cell transformation or a germ line transformation.

The present invention is further embodied by a method for providing an orally bio-available protein to a subject. The method includes the step of causing a plant to express at least a portion of the orally bio-available protein in at least some cells thereof. The method further includes the step of feeding at least a portion of the plant to the subject. The step of causing may be affected in a variety of ways, as detailed hereinabove for interferon, which is an example of an orally bio-available protein.

Methods disclosed herein represent a significant improvement upon the prior art because they do not require purification of interferon or other orally bio-available proteins from the plant.

The phrase “feeding at least a portion of the plant” as used in this specification and the accompanying claims should be construed in its broadest possible sense. Feeding may involve, for example, administration of fresh plant parts, dried plant parts, lyophilized plant parts, ground plant parts, powdered plant parts, juice extracted from plant parts, preserved (e.g. pickled or jellied) plant parts or plant parts subjected to any combination of processes including one of these processes.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Results detailed herein below in the examples section provide evidence that AGII can mediate the synthesis of a biologically active Interferon-alpha 2a in edible cucurbit fruit and leaves. Specifically, the highest activity of Interferon-alpha 2a was measured in cucumber and squash leaves (430,000 IU/gFW). This activity is similar to the interferon 2delta activity obtained in turnip when CaMV was used as a DNA virus vector (De Zoeten et al., 1989), and is equivalent to about 2 μg/gFW of active protein.

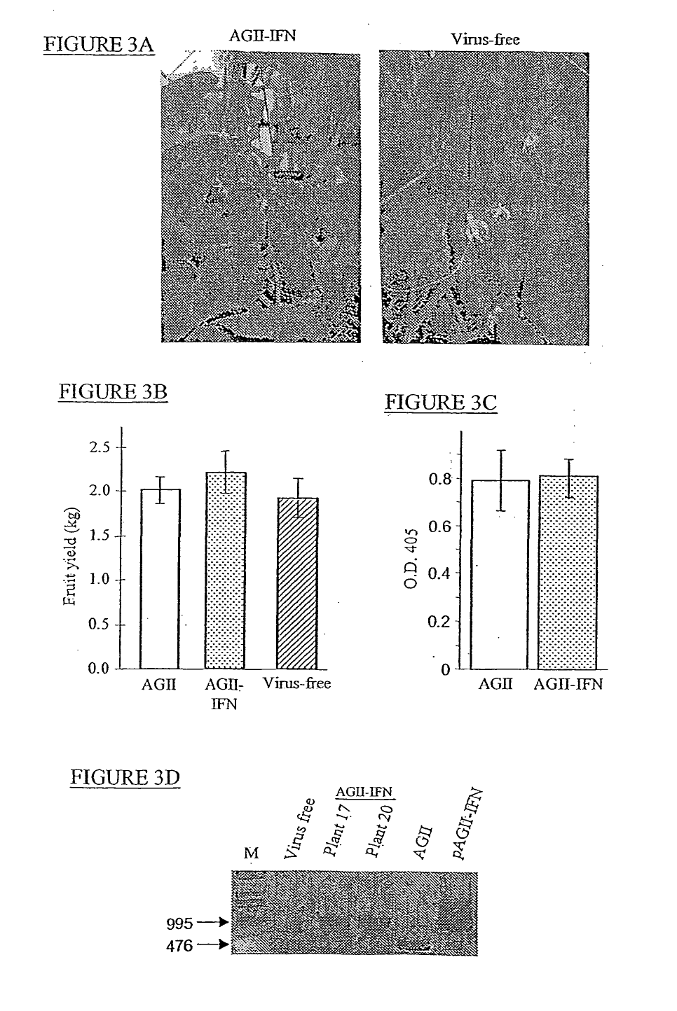

As AGII virus is not pathogenic, the amount and quality of fruit produced by AGII-interferon alpha 2a (AGII-IFN) infected cucumber plants was comparable to those of fruit from virus-free plants (FIG. 3A). Consistent with GFP expression in the fruits, IFN-2a activity measured in squash and cucumber was concentrated mainly in fruit embryonic tissue. Accumulation of AGII-IFN virions in fruits is a result of foreign gene expression mediated by viral replication and spread.

The activity of IFN-2a in cucumber leaves varied in accordance with the leaf developmental stage. In fully expanded leaves, weighing more than 10 g, the IFN-2a activity had declined while virus accumulation remained stable. This miscorrelation between AGII-IFN virion accumulation and foreign gene expression levels was probably due to a decrease of virus replication in mature tissue, together with a relatively turnover of interferon alpha-2a compared with the stability of the virion. The addition of seven amino acids at the carboxyl terminus of the IFN-2a in the AGII expression system did not affect its activity as, confirming the earlier observation of Petska that addition of amino acid residues to the termini of interferon did not affect its activity (Pestka et al, 1987). It is noteworthy that no IFN activity was lost when plant tissue was lyophilized.

Because orally administrated interferon was recently shown to be an efficient drug in animals (Marcus et al., 1999) and humans (Cummins et al., 1999), interferon, which is expressed in cucurbit fruit, may be administered orally to treat patients.

In summary, the present invention demonstrates the feasibility of using a potyvirus, for example the engineered attenuated AGII strain of ZYMV as an expression vector in cucurbits.

Thus, the primary advantage of the present invention, with respect to prior art is that the disclosed invention allows a significant reduction in the cost of production of interferon by eliminating the need for purification. Although in some cases edible plant parts may be subjected to simple processes such as grinding and drying to produce, for example, freeze dried fruit powder, the simplest embodiment of the invention involves giving the subject fresh produce to eat. In fact, distribution of plants to patients is within the scope of the claimed invention. Thus, according to its simplest embodiment, the present invention eliminates not only purification costs, but greatly reduces distribution, storage, shipping and packaging costs as well.

Further, the system and method of the present invention serve, to a large degree to eliminate concerns regarding toxic contaminants in the interferon preparation. This stems from the fact that, since the interferon is not prepared in bacteria, it is unlikely that bacterial toxins will be introduced during the manufacturing process. Similarly, there is no danger of introduction of human pathogens during the manufacturing process because human cell cultures are not employed. Thus, concerns about residual antibiotics, artificial preservatives and cell culture additives are also eliminated by practice of the present invention.

The present invention has all the inherent advantages of prior art oral administration methods including ease and comfort of administration. These factors make self-administration more acceptable to patients. Further, the plant cell wall can provide a slow release effect in vivo (Walmsley and Arntzen, 2000), perhaps making the present invention more suitable for use in certain clinical applications, for example Hepatitis C. The plant cell wall makes the present invention “saliva insoluble”, thereby differentiating it from the prior art. It is believed that the interferon of the present invention is protected from protease activity in the digestion system. As a result, the interferon is available for subsequent absorption in the gut wall, a possibility which is typically ruled out by prior art teachings.

Further, lyophilized plant material should be stable at room temperature without degradation of interferon contained therein. This serves to break the “cold chain” of transportation and storage, further reducing the final cost of each unit of delivered interferon. Further, this capacity for distribution without refrigeration makes practice of the present invention more feasible in less developed areas of the world. Such a consideration is crucial, for example in treatment of HCV and HIV.

Many proteins expressed in plant-virus systems in the prior art have proven to be unstable. Interferon alpha-2a, by contrast, has proven to be exceptionally stable.

The present invention offers several additional advantages relative to known plant bio-reactor systems. Yield is good because the vector is benign with respect to the host plant. Non-transmissibility by the natural aphid vector is easily achieved. The foreign gene, because it is not incorporated into the germ line of the plant, is not transmissible in seeds or pollen of the infected plant. In addition, transgenic plants require a significant development time due to requirements for screening and propagation. The present invention is free of this limit. Further, the present invention does not require delivery of viral RNA, relying instead upon delivery of a cDNA vector. This serves to significantly reduce the chance of accidental delivery to a plant because the cDNA expression vector is not an infectious virus.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney; R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); “Using Antibodies: A Laboratory Manual” (Ed Harlow, David Lane eds., Cold Spring Harbor Laboratory Press (1999)) all of which are incorpotaed by reference as if fully set forth herein. Other general references are provided throughout this document. The procedures therein are believed to be well known in the art and are provided for the convenience of the reader. All the information contained therein is incorporated herein by reference.

Additionally, the following methods were employed in performance of experiments described in examples presented hereinbelow:

Construction of a Non-aphid-transmissible AG

The aphid non-transmissible mutation was introduced in two steps. First, a PstI site was introduced in the NIa protease motif (DTVMLQ) within the NIb gene, between the encoding sequences of Leu and Glu (LQ), by site-directed mutagenesis on AG (Gal-On, 2000), with the partial clone pKS? sacI22 (7515-9591) used as a template. The resulting mutant clone was designated pKS? SacI-PstI. A nucleotide change, altering coat protein (CP) residue Ala⁹ to Thr, was then introduced by PCR on pKS? SacI-PstI as a template with an appropriate sense oligonucleotide 5′ATGCTGCAGTCAGGCACTCAGCCAACTGTGGCAGATACTGGAGCT-3′ containing the nucleotide change (bold). The mutated pKS?SacI-PstI SacI-MluI fragment was then introduced into SacI-MluI sites of AG to create AGI.

Construction of a Gene Insertion Cassette Between NIb and CP

A polylinker containing the restriction sites (PstI, ScaI, SpeI, NheI and SalI) with the NIa protease sequence (bold) was cloned by PCR with the oligonuclotide 5′CAGCTGCAGAGTACTAGTGCTAXGCGTCGACACTGTGATGCTCCA A -3′ on pKS? SacI-PstI used as a template. The PCR product was digested with PstI and YbaI (position 9461) and introduced into the appropriate sites within the pKS? SacI-PstI clone to create pKS? SacI-PstI-poly. pKS? SacI-PstI-poly SacI-MluI fragment was then introduced into SacI-MluI; sites of AGI to create AGII.

Insertion of Jellyfish Green Fluorescent Protein (GFP), uidA (Beta-Glucuronidase; GUS) Genes into the AGII Genome

The coding region of GFP (SEQ ID NO.: 15) was amplified by PCR, using sense and antisense oligonucleotides (SEQ ID Nos.; 17 and 18) that were both flanked by PstI sites. The amplified fragments were digested by PstI and cloned into the partial clone pKSΔSacI-PstI-poly. A similar cloning strategy was used for uicL4 (SEQ ID NO.: 16) using sense and antisense oligonucleotides (SEQ ID Nos.; 19 and 20), except that the antisense primer contained a flanking SalI site instead of PstI. Amplified PCR fragments were then digested by PstI and SalI and cloned into pKSΔSacI-PstI-poly. For all genes, pKSΔSacI-PstI-poly clones were double-digested by SacI/MluI, and the resulting fragment containing the foreign gene was cloned into AGII genome to create AGII-GFP and AGII-GUS.

Insertion of Human Interferon-Alpha 2a (IFN-2a) Genes into the AGII Genome

The coding region of IFN (SEQ ID NO.: 1) and CV-CP were amplified by PCR, using sense and antisense oligonucleotides (SEQ ID Nos.: 3 ands 4) that were both flanked by SalI sites. The amplified fragments were digested by SalI and cloned into the partial clone pKS? SacI-SalI-poly. Amplified PCR fragments were then digested by Sail and cloned into pKS? SacI-SalI-poly. For all genes, pKS? SacI-SalI-poly clones were double-digested by SacI/MluI, and the resulting fragment containing the IFN gene was cloned into AGII genome to create AGII-IFN.

Plant Growth, Inoculation and Symptom Evaluation

Commercial cultivars of squash (Cucrbita pepo L. cv. Ma'ayan) and cucumber (Cucumis sativus L. cv. Delila and cv. Muhasan) plants were grown in a growth chamber under continuous light at 23 degrees ° C. For test under industrial conditions, plants were grown in 20-1 pails with automatic irrigation and fertilization, in an insect-proof net-house. Seedlings were selected for experimental use when the cotyledons were fully expanded. Particle bombardment inoculation was performed with a handheld device, the handgun, with plasmid containing virus cDNA under the control of the cauliflower mosaic virus 35S promoter (Gal-On et al., 1997). Mild virus symptoms would be observable only in squash, as the AGII virus is symptomless on other cucurbits, therefore, it was chosen for testing the infectivity of various viral constructs. After bombardment or mechanical inoculation, squash seedlings were grown and examined daily for symptom development, and the first appearance of symptoms was recorded.

RT-PCR Analysis of Recombinant Virus Progeny

RT-PCR of viral progeny was conducted in a one-tube single-step method modified from Sellner et al. (1992). A 50-microliter volume was used containing the polylinker flanking primers 5-AGCTCCATACATAGCTGAGACA-3′ and 5′-TGGTTGAACCAAGAGGCGAA-3′ (SEQ ID NOs.: 5 and 6) in the following mixture: 1.5 mM MgCl₂; 125 μM dNTPs; 1× Sellner buffer: [10× Sellner buffer contains: 670 mM Tris-HCl; 170 mM (NH₄)₂SO₄; 10 mM beta-mercapto-ethanol; 2 mg/ml gelatin (Aldrich, calf skin 225 bloom); 60 μM EDTA pH 8.0 (Sellner et al., 1992)]; 100 ng of each specific primer; 2 units of Taq polymerase; 5 units of AMV-RT (Chimerex USA); 2-5 μg total RNA. RT-PCR cycles were as follows: 46 degrees C. 30 min; 94 degrees C. 2 min, followed by 33 cycles at 94 degrees C., 60 degrees C. and 72 degrees C., each of 30 s., and a final cycle of 5 min at 72 degrees C.

ELISA Assays for Evaluation of Viral Titer

Infected plant material was subjected to enzyme-linked immunosorbent assay (ELISA) with anti-ZYMV CP polyclonal antibody, as described previously by Antignus et al. (1989). The quantity of AGII-IFN was estimated by checking against a known amount of purified AGII virion in the ELISA plate.

IFN Activity Assay and Immunoblot Analysis

Plant tissue was collected, frozen in liquid N₂ and lyophilized for 24 h. Lyophilized tissue was ground by pestle and mortar and extracted in PBS with a ratio of 1:1-1.5 (dry weight tissue/per unit volume of PBS). One milliter of the homogenate was centrifuged for 10 min at 10,000 g in an Eppendorf minifuge, and the supernatant was used for ELISA, immunoblot analysis and interferon activity assay. IFN activity was assayed in 96-well microtiter plates by the inhibition of vesicular stomatitis virus cytopathic effect on human Wish (ATCC CCL-25) cells, as described previously (Rubinstein et al., 1981). Calibration standards of IFN were included in every plate. IFN activity was expressed in international units per milliliter (IU/ml), 2×10⁸ IU are equivalent to 1 mg IFN. For immunoblot (ECL, Amersham-Pharmacia Biotech, UK), extracts were separated on 15% SDS-PAGE and immunoblotted with an anti-IFN polyclonal antibody at 1:1000 dilution.

Assays of Impact of an Administered Therapeutic Agent on Colitis in a Mouse Model

An established mouse colitis model (Gotsman et al., 2001) was used to assess the effect of orally administered IFN from edible plant parts prepared according to the present invention.

Preparation of Plants Parts Containing Interferon for Oral Administration

Squash plants (cv. Guliver) were inoculated with a ZYMV-AGII-IFN cDNA at the seedling stage (4 days post emergence). Verification of infection was determined for each plant two weeks post infection by a DAS-ELISA with specific anti-ZYMV antibodies. Each plant was tested for interferon alpha biological activity 3 weeks post infection by a standard interferon alpha assay. Fruits were collected from AGII-IFN infected plants and AGII infected plants as a negative control 38 days after planting. Picked fruit was washed carefully, sliced, freeze-dried, and ground to a homogeneous powder. Powder was then extracted with phosphate saline buffer in a ratio of 1/7.6 (w/v) and the soluble fraction was collected and tested for its interferon alpha biological activity. Activity of 120,000 IU/ml interferon alpha was obtained. Similar procedure was done for negative control fruit. All interferon assays employed the National Institute of Health interferon alpha as a standard for activity.

Mice Experimental Groups

Five groups of mice (n=10) were studied. Mice from groups B, C, and D received orally either extract made from squash fruit expressing human interferon alpha 2a (1.875×10⁶ IFN IU/kg/dose) or from negative control squash (0 IFN IU/k-g/dose) fruit extract, every day for 14 days.

Clinical Assessment of Colitis and Macroscopic Score of Colitis

Diarrhea of mice was followed daily throughout the study. Colitis assessment was performed 10 days after colitis induction using standard parameters. Namely, mice were sacrificed and colon was removed. The percentage of the total colonic wall appearing injured and colon weight were recorded. Further, degree of colonic ulcerations; intestine and peritoneal adhesions; wall thickness; and degree of mucosal edema were assessed (Ilan et al., 2000). Each parameter was blindly graded on a scale from 0 (completely normal) to 4 (most severe) by two experienced examiners.

Grading of Histological Lesions

For histological evaluation of inflammation, distal colonic tissue (last 10 cm) was removed and fixed in 10% formaldehyde. Five paraffin sections from each mouse were then stained with hematoxylin-eosin according to standard techniques. The degree of inflammation on microscopic cross sections of the colon was be graded semiquantitatively from 0 to 4 (Ilan et al., 2000) (Grade 0: normal with no signs of inflammation; Grade 1: very low level of leukocyte infiltration; Grade 2: low level of leukocyte infiltration; Grade 3: high level of infiltration with high vascular density, and thickening of the bowel wall; and Grade 4: transmural infiltrates with loss of goblet cells, high vascular density, wall thickening, and disruption of normal bowel architecture.) Grading was performed blindly by two experienced pathologists.

Cytokines

Cytokines were measured in the serum by ELISA for IL4, IL10, IL12, and IFN gamma using Genzyme Diagnostics kits (Genzyme Diagnostics, Boston, Mass., USA) according to manufacturer's instructions. Serum levels were measured in all mice from all groups 14 days after starting the oral administration.

### Example 1

### Engineering AG to be an Aphid Non-Transmissible Virus

ZYMV, like other potyviruses, is naturally transmitted by aphids in a non-persistent manner (Desbiez and Lecoq, 1997). It has been shown that the CP Asp⁸Ala⁹Gly¹⁰ (DAG) motif is involved in transmission of ZYMV by aphids, and that mutation of alanine to threonine abolishes ZYMV transmission by aphids (Gal-On et al., 1992). A site-directed mutagenesis was performed to switch Ala⁹ residue to Thr (SEQ ID NOs.: 9 and 10) in the DAG motif of the AG CP, and the resultant mutant virus was designated AGI. Inoculation of AGI cDNA to squash plants resulted in infection indistinguishable from that caused by AG. The Ala-to-Thr alteration in the AGI progeny virus was verified by is RT-PCR and sequencing. An aphid transmission assay (Antignus et al., 1989) demonstrated that the AGI could not be transmitted by aphids, and this characteristic remained stable for prolonged propagation and several plant-to-plant mechanical inoculation passages. Based upon these encouraging results, AGI became the basis for further manipulation as detailed in hereinabove and used in example 2.

### Example 2

### Expression of Reporter Genes via AGII Vector in Various Cucurbits Tissues Including the Edible Fruit

To study AGII spread and localization of the expressed foreign protein in different organs, the bacterial uidA and jellyfish GFP genes were inserted into the NIb-CP site (FIG. 1B). Essentially 100% of squash plants inoculated by particle bombardment with the recombinant cDNA corresponding to AGII-GFP and AGII-GUS became infected. Typical vein clearing and mild mosaic symptoms appeared in AGII-GFP infected squash 5-7 dpi. For AGII-GUS, a 4-d delay of symptom appearance was observed.

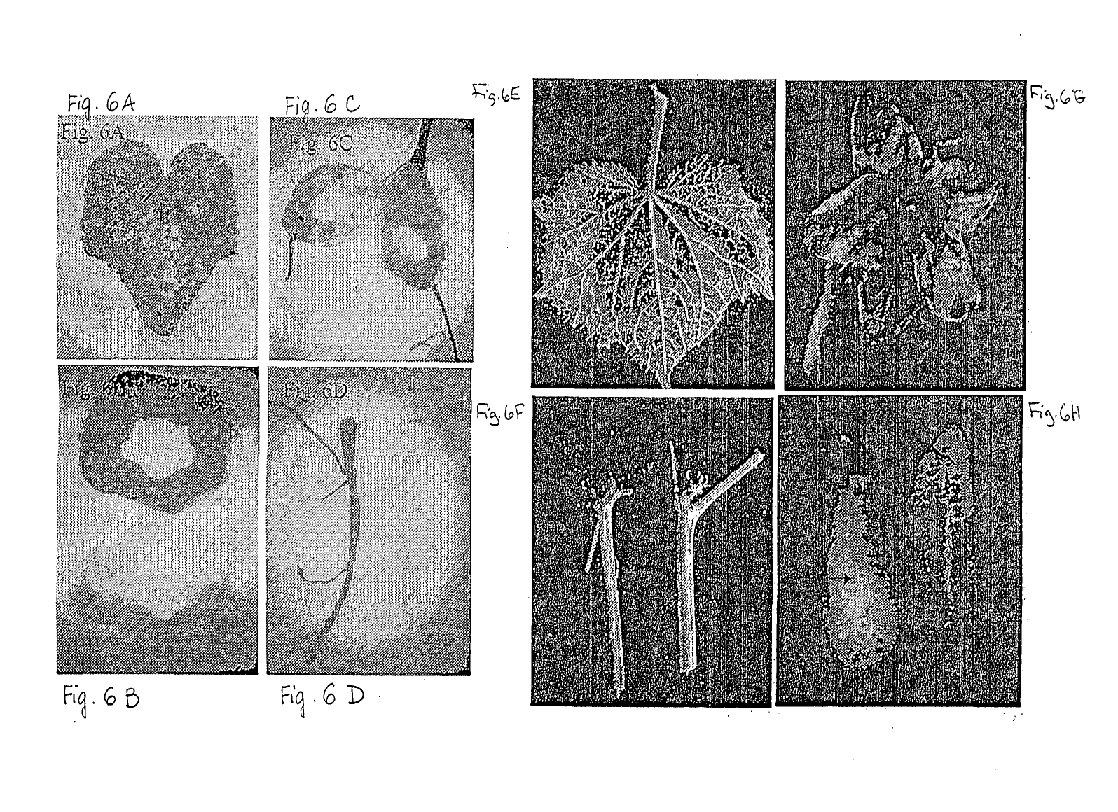

To follow the localization of foreign proteins expressed through the AGII virus vector, squash and cucumber seedlings were inoculated with AGE-GUS and AGII-GFP, respectively. AGII-GUS-infected squash was analyzed for GUS activity 15 dpi, and GUS staining was observed in leaves, stems and roots (FIGS. 6A-D). Distribution of GUS staining was not uniform in infected leaves, and staining concentrated around the major veins and neighboring cell clusters (FIG. 6A). Stems showed uniform staining, concentrated around the vascular tissue (FIGS. 6B-C). Interestingly, strong GUS staining was detected in adventives (FIG. 6C) and lateral roots (FIG. 6D). AGII-GFP infected cucumbers were analyzed for GFP by visualization under UV light. Green fluorescence was observed in AGII-GFP infected leaves, stems, flowers and fruit (FIG. 6E, F-right, G, H-left), indicating GFP expression in these organs. Similar fluorescence was not observed in identically developed organs infected with AGII (FIG. 6F-left, 6H-right); a non-uniform fluorescence was seen in leaves (FIG. 6E) and male flowers (FIG. 6G). In fruits, fluorescence was located mainly in the embryonic tissue and to a lesser degree in the peel layer or mesocarp (FIG. 6H-left).

These results indicate that a foreign gene expressed in plants according to the present invention is expressed in a variety of plant tissues including the fruit.

### Example 3

### Expression of a Biologically Active Human Interferon-Alpha 2a via AGII in Cucurbits

To quantify foreign gene expression in host plant organs, and to demonstrate the biotechnological potential of the AGII expression vector in cucurbits, we inserted the IFN coding sequence into the NIb-CP insertion site (FIGS. 1A and 1B). Plasmids containing AGII-IFN cDNA were inoculated on squash and cucumber plants yielding full infectivity. Symptoms similar to those elicited by the parental virus AGII were observed within 5-7 dpi. The presence of the IFN-2a gene within the AGII genome was verified by RT-PCR analysis of the progeny virus containing IFN-2a gene between NIb and CP.

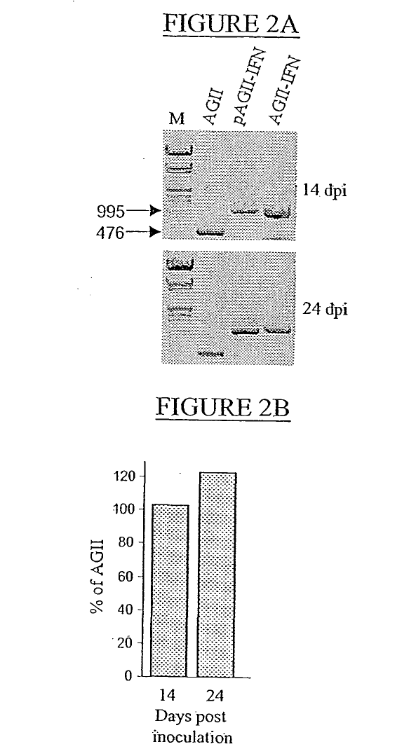

FIG. 2A is an RT-PCR analysis of progeny viral RNA. Total RNA was extracted from AGII-IFN systemically infected leaves, at 14 or 24 dpi, and subjected to RT-PCR with primers flanking the NIb-CP insertion site. Plasmids harboring cDNA of AGII-IFN (pAGII-IFN) were subjected to PCR as a control. Amplified products were then analyzed on an EtBr agarose gel (image negative is shown) The expected size (bp) of amplified fragment, containing the inserted gene and flanking 476 bp of AGII, is marked by an arrow. HindIII-EcoRI digested Lambda DNA was used as molecular weight marker (M).

FIG. 2B illustrates accumulation AGII-IFN in squash plants. Accumulation is expressed as the percentage of AGII accumulation (100%). The level of the virus was determined by DAS-ELISA and is the average of three independent samples taken from three independent plants. All samples were collected from developmentally equivalent leaves at the indicated dpi.

Thus, the IFN gene was maintained intact in the AGII genome at least 24 dpi. (FIG. 2A) and accumulated to similar levels as AGII (FIG. 2B). Moreover, stability of the IFN gene was maintained after six serial passages (at 3-week intervals) from plant to plant.

Commercial cultivars of squash (Cucurbita pepo L. cv. Ma'ayan) and parthenocarpic cucumber (Cucumis sativus L. cv. Muhasan) seedlings were infected by sap inoculation of AGII-IFN (eight plants) or AGII (four plants). As a control, non-infected plants (four plants) were included. Plants were grown vertically in a semi-industrial net house under automatic irrigation and fertilization. FIG. 3A includes photographs of AGII-IFN-infected and virus-free plants, which were taken 45 days after seedling inoculation. No difference is apparent. Plant infection was verified by DAS-ELISA. The effects of AGII-IFN infection on plant growth and development were evaluated by monitoring the plant phenotype and symptom expression, and by estimating the crop yield. During the growth period, cucumber plants infected with AGII-IFN developed normally. AGII-IFN plants did not show any visible symptoms on their leaves or fruit, and were phenotypically indistinguishable from virus-free plants (FIG. 3A). Infected squash plants also developed normally, showing only mild diffused mosaic symptoms on their leaves, and no symptoms on their fruits (not pictured). Crop yield was measured by collecting marketable cucumber fruits (about 60 g each) for a period of 1 month, beginning 3 weeks post inoculation. FIG. 3B is a histogram comparing cucumber yield among virus-free plants, and AGII- and AGII-IFN-infected plants. Fruits (average size of 60 g) were collected from plants during 1 month. Data are given as the mean±SD of three or four independent plants. A yield of about 2 kg of fruit per plant was obtained in virus-free plants (FIG. 3B), and a comparable yield was obtained in AGII-IFN and AGII inoculated plants (FIG. 3B).

FIG. 3C is a histogram showing accumulation of AGII and AGII-IFN viruses in cucumber plants. The level of virus was determined by DAS-ELISA in four samples from independent plants. All samples were collected from developmentally equivalent leaves at 45 dpi. Similar levels of virus accumulation were measured in the leaves of these plants (FIG. 3C), demonstrating that virus infection did not affect fruit production.

FIG. 3D is an RT-PCR analysis of progeny viral RNA. Total RNA was extracted from leaves of recombinant virus (as indicated) infected plants or from virus-free plants, and subjected to RT-PCR with primers flanking the IFN insertion point. A plasmid harboring AGII-IFN cDNA (pAGII-IFN) was subjected to PCR as a control. The expected size (bp) of the fragment with (995) or without (476) the IFN is marked by an arrow. HindIII-EcoRI-digested Lambda DNA was used as a molecular weight marker (M); it is noteworthy that the IFN gene within AGII-IFN remained intact in tested plants (plants numbers 17 and 20 are shown), even 2 months post inoculation, as confirmed by RT-PCR (FIG. 3D).

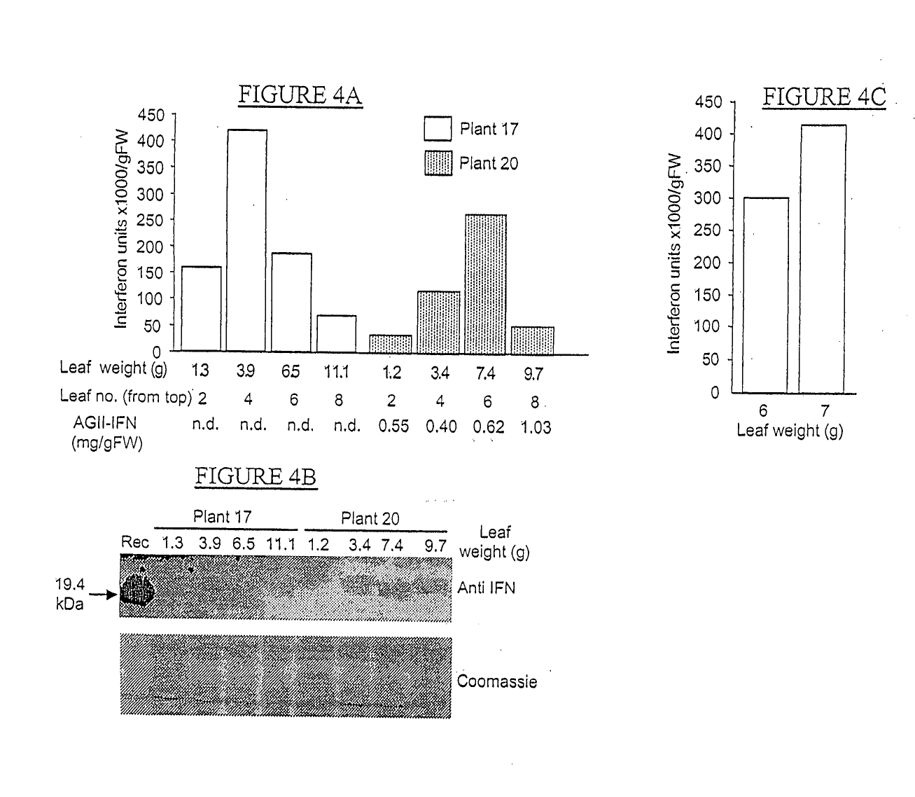

FIG. 4A is a histogram of IFN activity measured in leaves of AGII-IFN-inoculated cucumber at 60 dpi. The values were obtained after subtracting the background activity (of AGE-infected cucumber). Data are given as the mean±SD of three independent measurements. Tested leaf developmental stage (weight and position from the top) and AGII-IFN virus amount are presented below the histogram. n.d.=not determined. Infected leaves from the above cucumber (representative plants 17 and 20) and squash plants were analyzed for IFN activity at 60 and 30 dpi, respectively. Activities of 157×10³ and 34×10³ IU per gram fresh weight (gFW) were measured in young leaves (2^{nd} leaf; FIG. 4A). Much higher IFN activity was found in older leaves (4^{th}-6^{th} leaves; FIG. 4A). However, after leaves had fully expanded (8^{th} leaf), a sharp decrease in IFN activity occurred (FIG. 4A). An average activity of 21×10³ IU/gFW was measured in stems.

FIG. 4B is an Immunoblot analysis of samples tested in FIG. 4A. Soluble protein extracts (70 μg) were analyzed by using anti-IFN polyclonal antibody. Recombinant IFN (Rec, 4 ng) was used as a control for gel mobility. Immunoblot analysis of samples which had been analyzed for interferon revealed the presence of a protein band that reacted with an anti-IFN antibody. Moreover, band intensity correlated with the level of IFN activity, indicating that this band represented IFN (FIG. 4B). As predicted, this band exhibited a slightly slower gel mobility than that of recombinant hIFN-2a due to the addition of eight amino acid residues to the IFN sequence (FIG. 1B).

FIG. 4C illustrates IFN activity measured in leaves of AGII-IFN inoculated squash at 30 dpi. The values obtained after subtracting the background activity (of AGII-infected squash). Data are given as the mean±SD of three independent measurements. In squash, IFN activity in young leaves (4th from the top, FIG. 4C) was comparable with that in those of cucumber (FIG. 4A). No activity was found in leaves of control plants. To correlate between virus accumulation and protein expression in leaves, the amount of AGII CP in the tested leaves was measured by quantitative DAS-ELISA (FIG. 4A, below histogram). An increase in the amount of AGII CP was measured as the leaf matured. No correlation was obtained between CP accumulation and the biological activity of IFN. This was especially prominent in fully expanded leaves that contained the greatest amount of AGII CP and exhibited the lowest IFN activity (FIG. 4A).

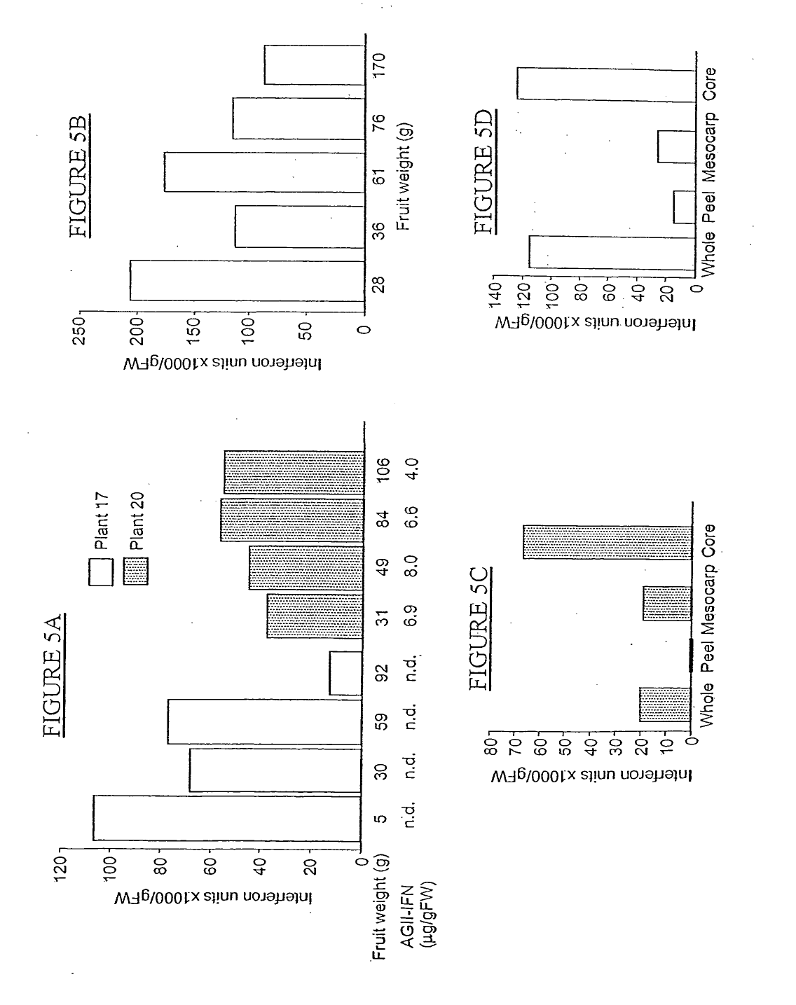

FIGS. 5A and B depict IFN activity found in fruit extracts from AGII-IFN inoculated cucumber (FIG. 5A) or squash (FIG. 5B) plants, 60 or 30 dpi, respectively. The values obtained after subtracting the background activity (of AGII-infected plants). Data are given as the mean±SD of three independent measurements. Tested fruit developmental stage (weight) and AGII-IFN virus amount are presented below the histogram. n.d.=not determined.

The IFN activity measured in fruits from the same cucumber and squash plants (FIGS. 5A and 5B) was two-to fourfold lower than activity in leaves (FIGS. 4A and 4C) of the same plants. The highest activity was found in the youngest immature fruits of both cucumber and squash (FIGS. 5A and 5B). On average, a twofold greater increase in IFN activity was measured in squash fruits than in those of cucumber (FIGS. 5A and 5B). Accumulation of AGII CP in cucumber fruits was two orders of magnitude less than in leaves, which is consistent with the IFN activity difference between the two organs.

FIGS. 5C and D depict IFN activity found in fruit parts from AGII-IFN inoculated cucumber plants 20 (FIG. 5C) or squash (FIG. 5D) 60 or 30 dpi, respectively. The values obtained after subtracting the background activity of AGII-infected fruit. Data are given as the mean±SD of three independent measurements.

Interestingly, analysis of IFN activity in cucumber and squash fruit parts shows that most of the activity was located in the fruit placental tissue and/or embryonic tissue (core) and much lower in the mesocarp and peel layer (FIGS. 5C and 5D).

### Example 4

### Expression of a Biologically Active Human Interferon-Alpha 2a via AGII in Different Cucurbit Cultivars

In order to establish that interferon is easily produced in a variety of agriculturally important cultivars, experiments were carried out in commercial cultivars of zucchini squash and cucumber. Results are summarized in table 1. Levels of interferon expression were high in all tested cultivars.

TABLE 1
Interferon alpha 2a activity in fruit from various
cucurbit cultivars

| | | Interferon |
|---|---|---|
| | | alpha 2a |
| Species | Cultivar | IU/gFW^{a} |
| Cucumber | Muhasan | 11534 |
| (cucumis sativus) | IV-40 | 8759 |
| | Sarig | 8428 |
| Zucchini squash | Marrow | 13693 |
| (cucurbita pepo) | Ma'ayan | 22939 |
| | Cocozelle | 24977 |
| | XPS136 | 15690 |
| | XPS159 | 18792 |
| | Goldy | 12957 |
| | Scaloppini | 18779 |
| | Crookneck | 14238 |
| | Zucchini | 17909 |
| | Erlica | 22316 |
| | Straightneck | 20425 |
| | Nano-Verde | 57142 |
| | Gulliver | 137500 |

^{a}Average activity measured from at least three independent fruits.

### Example 5

### Effect of Oral Administration of Human Interferon Alpha 2a Produced in Squash on Experimental Colitis in Mice

In order to measure the effect of interferon alpha 2a produced in plants on colitis, the TNBS mouse model of colitis was employed. The model is essentially as described in Gotsmann et al. (2001) and in Ilan et al. (2000). Briefly, mice were normal inbred females mice maintained on standard laboratory chow and kept in 12 hr light/dark cycles. Colitis was induced by intracolonic instillation of trinitrobenzene sulfonic acid (TNBs). Treated mice were dosed orally with extract of squash fruit expressing interferon alpha 2a for 14 days following colitis induction. As a control, colitis induced mice received either similar amounts of extract from squash fruit not expressing interferon alpha 2a or bovine serum albumin. Colitis was assessed in each group by standard clinical, macroscopic and microscopic scores. Serum cytokine secretion was determined by ELISA.

Evaluation of the effects of tolerance induction on experimental colitis was accomplished by assessing level of diarrhea, macroscopic scoring of colitis, cytokine levels and grading of histological lesions. Results are summarized in table 2.

Oral administration of either squash extract or squash extract containing interferon alpha 2a to mice not induced to colitis had no adverse impact on their health status (groups B and C). However, oral administration of extract from is squash fruit expressing interferon alpha 2a to mice induced to colitis (group D) markedly ameliorated their experimental colitis. These mice of group D gained weight, had less severe diarrhea, and showed markedly improved macroscopic and microscopic parameters of colitis. IFNγ levels decreased and IL10 levels increased in these mice as compared with mice induced to colitis and not given squash extract expressing interferon alpha 2a (group E).

In summary, this experiment demonstrates that oral administration of squash extract from fruit expressing human interferon alpha 2a exerted a positive impact on the intestine of colitis induced mice. This indicates that the interferon was absorbed in the digestive tract after swallowing in contrast to prior art teachings. Whether the observed effect is systemic or local, it represents a significant improvement in the applicability of oral interferon treatment to clinical medicine.

TABLE 2
Effect of oral administration of extracts from squash fruit
expressing interferon alpha 2a in a mouse colitis model

| | Colitis | | Microscopic | Macroscopic | | | | |
|---|---|---|---|---|---|---|---|---|
| Group | induced | Treatment | score: | score: | IFNγ | IL4 | IL10 | IL12 |
| A | NO | NONE | 0 | 0 | 160 | 39.3 | 90 | — |
| B | NO | Extract with | 0 | 0 | 101 | 14.5 | 76.2 | 223 |
| | | interferon | | | | | | |
| | | alpha 2a | | | | | | |
| C | NO | Extract with | 0 | 0 | 270 | — | 35.4 | 190 |
| | | out | | | | | | |
| | | interferon | | | | | | |
| D | YES | Extract with | 1.65 | 1.4 | 134 | 11.5 | 65.37 | 230 |
| | | interferon | | | | | | |
| | | alpha 2a | | | | | | |
| E | YES | NONE | 2.4 | 2.5 | 250 | 5 | 6 | — |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

Antignus, Y., Raccah; B., Gal-On, A., Cohen, S., 1989. Biological and serological characterization of zucchini yellow mosaic virus and watermelon mosaic virus-2 isolates in Israel. Phytoparasitica 17, 289-287.

Arazi, T., G. Slutzky, Y. Shiboleth, Y. Wang, M. Rubinstein, S. Barak, J. Yang, and A. Gal-On. 2001. Engineering zucchini yellow mosaic potyvirus as a non-pathogenic vector for expression of heterologous proteins in cucurbits. J. Biotech. 87, 67-82.

Bocci, V., 1999. The Oropharyngeal Delivery of Interferons: Where Are We and Where Do We Need to Go?, J. Interferon Cytokine Res. 19:859-861.

Carrington, J. C., Cary, S. M., Dougherty, W. G., 1988. Mutational analysis of tobacco etch virus polyprotein processing: cis and trans proteolytic activities of polyproteins containing the 49-kilodalton proteinase. J. Virol. 62, 2313-2320.

Carrington, J. C., Cary, S. M., Parks, T. D., Dougherty, W. G., 1989. A second proteinase encoded by a plant potyvirus genome. EMBO J. 8, 365-370.

Chapman, S., Kavanagh, T., Baulcombe, D. C., 1992. Potato virus X as a vector for gene expression in plants. Plant J. 2, 549-557.

Choi, I. R., Stenger, D. R., Morris, T. J., French, R., 2000. A plant virus vector for systemic expression of foreign genes in cereals. Plant J. 23, 547-555.

Cummins, J. M., Beilharz, M. W., Krakowka, S., 1999. Oral use of interferon. J. Interferon Cytokine Res. 19, 853-857.

Desbiez, C., Lecoq, H., 1997. Zucchini yellow mosaic virus. Plant Pathol. 46, 809-829.

De Zoeten, G. A., Penswick, J. R., Horisberger, Mi. A., Ahl, P., Schultze, M., Hohn, T., 1989. The expression, localization, and effect of a human interferon in plants. Virology 172, 213-222.

Dolja, V. V., McBride, B. J., Carrington, J. C., 1992. Tagging of plant potyvirus replication and movement by insertion of b-glucuronidase into the viral polyprotein. Proc. Natl. Acad. Sci. USA 89, 10208-10212.

Dolja, V. V., Herndon, K. L., Pirone T. P., Carrington, J. C., 1993. Spontaneous mutagenesis of a plant potyvirus genome after insertion of a foreign gene. J. Virol. 67, 5968-5975.

Dolja, V. V., Hong, J., Keller, K. E., Martin, R. R., Peremyslov, V. V., 1997. Suppression of potyvirus infection by coexpressed closterovirus protein. Virology 234, 243-252.

Fleischmann, W. R. JR. and Koren, S., 1999. Systemic Effects of Orally Administered Interferons and Interleukin-2, J. Interferon Cytokine Res. 19:829-839.

Gal-On, A., Antignus, Y., Rosner, A., Raccah, B., 1992. A coat protein gene mutation of zucchini yellow mosaic virus restored aphid transmissibility but has no effect on multiplication. J. Gen. Virol. 73, 2183-2187.

Gal-On, A., Meiri, A., Elman, C., Gray, D. J., Gaba, V., 1997. Simple hand-held devices for the efficient infection of plants with viral-encoding constructs by particle bombardment. J. Virol. Methods 64, 103-110.

Gal-On, A., 2000. A point mutation in the FRNK motif of the potyvirus HC-Pro gene alters symptom expression in cucurbits and exhibits protection against the severe homologous virus. Phytopathology 90, 467-473.

German-Retana, S., Candresse, T., Alias, E., Delbos, R. P., and Le Gall, O., 2000. Effects of green fluorescent protein or beta-glucuronidase tagging on the accumulation and pathogenicity of a resistance-breaking Lettuce mosaic virus isolate in susceptible and resistant lettuce cultivars. Mol. Plant Microbe Interact. 13, 316-324.

Gopinath, K., Wellink, J., Porta, C., Taylor, K. M., Lomonossoff, G. P., van Kammen, A., 2000. Engineering cowpea mosaic virus RNA-2 into a vector to express heterologous proteins in plants. Virology 267, 159-173.

Gotsman 1, Shlomai A, Alper R, Rabbani E, Engelhardt D, Ilan Y., 2001. Amelioration of immune-mediated experimental colitis: tolerance induction in the presence of preexisting immunity and surrogate antigen bystander effect. J. Pharmacol. Exp. Ther. 297(3):926-32.

Guo, H. S., Lopez-Moya, J. J., Garcia, J. A., 1998. Susceptibility to recombination rearrangements of a chimeric plum pox potyvirus genome after insertion of a foreign gene. Virus Res. 57, 183-195.

Hamamoto, H., Suglyama, Y., Nakagawa, N., Hashida, E., Matsunaga, Y., Takemoto, S., Watanabe, Y. Okada, Y., 1993. A new tobacco mosaic virus vector and its use for the systemic production of angiotensin-I-converting enzyme inhibitor in transgenic tobacco and tomato. Bio/Technology 11, 930-932.

Hammond-Kosack, K. E., Staskawicz, B. J., Jones, J. D. G., Baulcombe, D. C., 1995. Function expression of a fungal avirulence gene from a modified potato virus X genome. Mol. Plant-Microbe. Interact 8, 181-185.

Hendy, S, Chen, Z. C., Barker, H., Santa-Cruz, S., Chapman, S., Torrance, L., Cockburn, W., Whitelam, G. C., 1999. Rapid production of single-chain Fv fragments in plants using a potato virus X episomal vector. J. Immunol. Meth. 231, 137-146.

Ilan Y, Weksler-Zangen S, Ben-Horin S, Diment J, Sauter B, Rabbani E, Engelhardt D, Chowdhury N R, Chowdhury J R, Goldin E., 2000. Treatment of experimental colitis by oral tolerance induction: a central role for suppressor lymphocytes. Am J Gastroenterol. 95, 966-973.

Kumagai, M. H., Turpen, T. H., Weinzettl, N., della-Cioppa, G., Turpen, A. M., Donson, J., Hilf, M. E., Grantham, G. L., Dawson, W. O., Chow, T. P., Piatak, M., Jr., Grill, L. K., 1993. Rapid, high-level expression of biologically active a-trichosanthin in transfected plants by an RNA viral vector. Proc. Natl. Acad. Sci. USA 90, 427-430.

Kumagai, M. H, Donson, J., della-Cioppa, G., Grill, L. K., 2000. Rapid, high-level expression of glycosylated rice alpha-amylase in transfected plants by an RNA viral vector. Gene. 245, 169-174.

Marcus, P. I., van der Heide, L., Sekellick, M. J., 1999. Interferon action on avian viruses. 1. Oral administration of chicken interferon-alpha ameliorates Newcastle disease. J. Interferon Cytokine. Res. 19, 881-885.

Masuta, C., Yamana, T., Tacahashi, Y., Uyeda, I., Sato, M., Ueda, S., Matsumura, T., 2000. Development of clover yellow vein virus as an efficient stable gene-expression system for legume species. Plant J. 23, 539-546.

Matthews, R. E. F. 1991. Plant Virology. 3 ed. Academic Press, New York.

McCormick, A. A., Kumagai, M. H., Hanley, K., Turpen, T. H., Hakim, I, Grill, L. K., Tuse, D, Levy, S., Levy, R., 1999. Rapid production of specific vaccines for lymphoma by expression of the tumor-derived single-chain Fv epitopes in tobacco plants. Proc. Natl. Acad. Sci. USA 96, 703-708.

Pestka, S., Langer, J. A., Zoon, K. C., Samuel, C. E., 1987. Interferons and their actions. Annu. Rev. Biochem. 56, 727-777.

Porta, C., Lomonossoff, G. P., 1996. Use of viral replicons for the expression of genes in plants. Mol. Biotechnol. 5, 209-221.

Revers, F., Gall, O. L., Candresse, T., Maule, A. J., 1999. New advances in understanding the molecular biology of plant potyvirus interactions. Mol. Plant-Microbe. Interact 12, 367-376.

Riechmann, J. L., Lain, S. Garcia, J. A., 1992. Highlights and prospects of potyvirus molecular biology. J. Gen. Virol. 73, 1-16.

Rommens, C. M., Salmeron, J. M., Baulcombe, D. C. Staskawicz, B. J., 1995. Use of a gene expression system based on potato virus X to rapidly identify and characterize a tomato pto homolog that controls fenthion sensitivity. Plant Cell 7, 249-257.

Rubinstein, S., Familletti, P. C., Pestka, S., 1981. Convenient assay for interferons. J. Virol. 37, 755-758.

Sablowski, R. W. M., Baulcombe, D. C., Bevan, M., 1995. Expression of a flower-specific Myb protein in leaf cells using a viral vector causes ectopic activation of a target promoter. Proc. Natl. Acad. Sci. USA 92, 6901-6905.

Scholthof, H. B., Scholthof, K-B. G., Jackson, A., 1996. Plant virus gene vectors of transient expression of foreign proteins in plants. Ann. Rev. Phytopathal. 34, 299-323.

Sellner, L. N., Coelen, R. J., Mackenzie, J. S., 1992. A one-tube, one manipulation RT-PCR reaction for detection of Ross River virus. J. Virol. Meth. 40, 255-263.

Ship, J. A., Fox, P., Michalek, J. E., Cummins, M. J., Richards, A. B. and the IFN Protocol Study Group, 1999. Treatment of Primary Sjögren's Syndrome with Low-Dose Natural Human Interferon-a Administered by the Oral Mucosal Route: A Phase II Clinical Trial, J. Interferon Cytokine Res. 19:943-951.

Shukla, D. D., Ward, C. W., Brunt, A. A., 1994. The Potyviridae. CABI Wallingford.

Takamatsu, N., Ishikawa, M., Meshi, T., Okada, Y., 1987. Expression of bacterial chloramphenicol acetyltransferase gene in tobacco plants mediated by TMV-RNA. EMBO J. 6, 307-311.

Tompkins, W., 1999. Immunomodulation and Therapeutic Effects of the Oral Use of Interferon-a: Mechanism of Action, J. of Interferon and Cytokine Res. 19:817-828.

Valerie, E., Spall, M., Shanks, M., Lomonossoff, G. P., 1997. Polyprotein processing as a strategy for gene expression in RNA viruses. Seminars in Virology 8, 15-23.

Verchot, J., Koonin, E. V., Carrington, J. C., 1991. The 35-kDa protein from the N-terminus of a polyviral polyprotein functions as a third virus-encoded proteinase. Virology 185, 527-535.

Walmsley, A. M., Arntzen, C. J. 2000. Plants for delivery of edible vaccines. Curr. Opin. Biotechnol. 11, 126-9.

Walter, M. R., Bordens, R., Nagabhushan, T. L., Williams, B. R., Herberman, R. B., Dinarello, C. A., Borden, E. C., Trotta, P. P., Pestka, S., Pfeffer, L. M., 1998. Review of recent developments in the molecular characterization of recombinant alfa interferons on the 40th anniversary of the discovery of interferon. Cancer Biother. Radiopharm. 13, 143-54.

Zhang, G., Leung, C., Murdin, L., Rovinski, B., White, K. A., 2000. In planta expression of HIV-1 p24 protein using an RNA plant virus-based expression vector. Mol. Biotechnol. 14, 99-107.

What is claimed is:

1. A system for providing supplemental interferon to a subject, the system comprising:
(a) a viral vector, said vector designed and constructed to be capable of infecting a plant and expressing at least a portion of an interferon gene therein; and
(b) said plant, at least a portion of said plant being edible by the subject;
wherein a gene product of said at least a portion of an interferon gene is bioavailable to the subject consuming said at least a portion of said plant.

2. The system of claim 1, wherein said viral vector is a potyvirus vector.

3. The system of claim 2, wherein said potyvirus is zucchini yellow mosaic virus (ZYMV).

4. The system of claim 3, wherein said ZYMV is an attenuated strain containing a mutation as listed in SEQ ID NOs.: 7 and 8.

5. The system of claim 1, wherein said at least a portion of an interferon gene comprises a mammalian interferon gene sequence.

6. The system of claim 5, wherein said mammalian interferon gene sequence comprises at least a portion of a human interferon gene sequence.

7. The system of claim 6, wherein said human interferon gene sequence is selected from the group consisting of interferon alpha 2a (SEQ ID NO.: 1) and any gene at least 85% homologous thereto as analyzed by the FastA program.

8. The system of claim 6, wherein said human interferon gene sequence is selected from the group consisting of interferon beta (SEQ ID NO.: 11) of interferon gamma (SEQ ID NO.: 13) and any gene at least 85% homologous to either of said interferon genes as analyzed by the FastA program.

9. The system of claim 1, wherein said vector expresses at least a portion of a protein selected from the group consisting of the interferon alpha 2a gene product (SEQ ID NO.: 2) and any protein at least 85% homologous thereto as analyzed by the FastA program.

10. The system of claim 1, wherein said vector expresses at least a portion of a protein selected from the group consisting of the interferon beta gene product (SEQ ID NO.: 12), the interferon gamma gene product (SEQ ID NO.: 14) and any protein at least 85% homologous to either of said interferon gene products as analyzed by the FastA program.

11. The system of claim 1, wherein transmissibility of said viral vector from said plant to a second plant is prevented by a mutation in said viral vector.

12. A system for providing supplemental interferon to a subject, the system comprising:
(a) a DNA sequence designed and constructed to be capable of expressing at least a portion of an interferon gene in a plant; and
(ii) said plant, at least a portion of said plant being edible by the subject and said plant susceptible to transformation by said DNA sequence;
wherein a gene product of said at least a portion of an interferon gene is bioavailable to the subject consuming said at least a portion of said plant.

13. The system of claim 12, further comprising a means for introducing said DNA sequence into at least one cell of said plant, thereby transforming said cell.

14. The system of claim 12, wherein said DNA sequence comprises a left border and a right border of agrobacterium T-DNA.

15. The system of claim 12, wherein said at least a portion of an interferon gene comprises a mammalian interferon gene sequence.

16. The system of claim 15, wherein said mammalian interferon gene sequence comprises at least a portion of a human interferon gene sequence.

17. The system of claim 16, wherein said human interferon gene sequence is selected from the group consisting of interferon alpha 2a (SEQ ID NO.: 1) and any gene at least 85% homologous thereto as analyzed by the FastA program.

18. The system of claim 14, wherein said human interferon gene sequence is selected from the group consisting of interferon beta (SEQ ID NO.: 11) of interferon gamma (SEQ ID NO.: 13) and any gene at least 85% homologous to either of said interferon genes as analyzed by the FastA program.

19. The system of claim 12, wherein said vector expresses at least a portion of a protein selected from the group consisting of the interferon alpha 2a gene product (SEQ ID NO.: 2) and any protein at least 85% homologous thereto as analyzed by the FastA program.

20. The system of claim 12, wherein said vector expresses at least a portion of a protein selected from the group consisting of the interferon beta gene product (SEQ ID NO.: 12), the interferon gamma gene product (SEQ ID NO.: 14) and any protein at least 85% homologous to either of said interferon gene products as analyzed by the FastA program.

21. A method for providing supplemental interferon to a subject, the method comprising the steps of:
(a) causing a plant to express at least a portion of an interferon gene in at least some cells thereof; and
(b) feeding at least a portion of said plant to the subject.

22. The method of claim 21, wherein said step of causing is accomplished by an action selected from the group consisting of:
(i) infecting at least one cell of said plant with a viral vector, said viral vector designed and constructed to be capable of expressing at least a portion of an interferon gene therein; and
(ii) transforming at least one cell of said plant with a DNA sequence designed and constructed to be capable of expressing at least a portion of an interferon gene therein.

23. The method of claim 21, wherein said at least a portion of an interferon gene comprises a mammalian interferon gene sequence.

24. The method of claim 23, wherein said mammalian interferon gene sequence comprises at least a portion of a human interferon gene sequence.

25. The method of claim 24, wherein said human interferon gene sequence is selected from the group consisting of interferon alpha 2a (SEQ ID NO.: 1) and any gene at least 85% homologous thereto as analyzed by the FastA program.

26. The method of claim 24, wherein said human interferon gene sequence is selected from the group consisting of interferon beta (SEQ ID NO.: 11) of interferon gamma (SEQ ID NO.: 13) and any gene at least 85% homologous to either of said interferon genes as analyzed by the FastA program.

27. The method of claim 21, wherein said step of causing a plant to express includes expression of at least a portion of a protein selected from the group consisting of the interferon alpha 2a gene product (SEQ ID NO.: 2) and any protein at least 85% homologous thereto as analyzed by the FastA program.

28. The method of claim 21, wherein said step of causing a plant to express includes expression of at least portion of a protein selected from the group consisting of the interferon beta gene product (SEQ ID NO.: 12), the interferon gamma gene product (SEQ ID NO.: 14) and any protein at least 85% homologous to either of said interferon gene products as analyzed by the FastA program.

29. A method for providing an orally bio-available protein to a subject, the method comprising the steps of:
(a) causing a plant to express at least a portion of the orally bio-available protein in at least some cells thereof; and
(b) feeding at least a portion of said plant to the subject.

30. The method of claim 29, wherein said step of causing is accomplished by an action selected from the group consisting of:
(a) infecting at least one cell of said plant with a viral vector, said viral vector designed and constructed to be capable of expressing at least a portion of a gene encoding the orally bio-available protein therein; and
(b) transforming at least one cell of said plant with a DNA sequence designed and constructed to be capable of expressing at least a portion of a gene encoding the orally bio-available protein therein.


SEQUENCE LISTING (20 sequences)

SEQ ID NO: 1 - DNA, 498 bp, Homo sapiens
atgtgtgatctgccgcagactcactctctgggttctcgtcgtactctgatgctgctggctcagatgcgtcgtatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaa

SEQ ID NO: 2 - protein, 166 aa, Homo sapiens [sequence omitted]

SEQ ID NO: 3 - DNA, 34 bp, Artificial
  synthetic oligonucleotide primer
gctgcagtcatgtgatctgccgcagactcactct

SEQ ID NO: 4 - DNA, 30 bp, artificial
  oligonucleotide primer
cagtgtcgacttccttacttcttaaacttt

SEQ ID NO: 5 - DNA, 22 bp, Artificial
  synthetic oligonucleotide primer
agctccatacatagctgagaca

SEQ ID NO: 6 - DNA, 20 bp, Artificial
  synthetic oligonucleotide primer
tggttgaaccaagaggcgaa

SEQ ID NO: 7 - DNA, 1359 bp, Zucchini yellow mosaic virus
tcacaaccggaagttcagttcttccaaggatggcgacgaatgtttgacaagtttaggcccagcctagatcatgtgtgcaaagttgaccacaacaacgaggaatgtggtgagttggcagcaatcttttgtcaggctctattcccagtagtgaaactatcgtgccaaacatgcagagaaaagcttagtagagttagcttcgaggaattcaaagactctttgaacgcaaactttattatccacaaggatgaatgggatagtttcaaggaaggctctcattacgataatattttcaaattgatcaaagtggcaacacaggctactcagaatctcaagctctcatctgaagttatgaagttagttcagaaccacacaagcactcacatgaagcaaatacaagacatcaacaaggcgctcatgaaaggttcattggttacgcaagacgaattggacttagctttgaaacagcttcttgaaatgactcagtggtttaagaaccacatgcatctgactggtgaggaggcattgaaaatgttcataaataagcgctctagcaaggccatgataaatcctagccttctatgtgacaaccaattggacaaaaatgaaattttgtttggggagaaagagatacattccaagcgattattcaagaacttcttcgaagaagtataccagcgaaggatatacgaagtacgtagtgcgaactttccaaatggtactcgtaagttggccataggctcattgattgtaccactcaatttggatagggcacgcactgcactacttggagagagtattgagaagaagccactcacatcagcgtgtgtctcccaacagaatggaaattatatacactcatgctgctgtgtaacgatggatgatggaaccccgatgtactcagagcttaagagcccgacgaagaggcatctagttataggagcttctggtgatccaaagtacattgatctgccagcatctgaggcagaacgcatgtatatagcaaaagaaggttattgctatctcaatattttcctcgcaatgcttgtgaatgttaatgagaacgaagcaaaggatttcaccaaaatgattcgtgatgttttgatccccatgcttgggcagtggccttcattgatggatgttgcaactgcagcatatattctaggtgtattccatcctgaaacgcgatgcgctgaattacccaggatccttgttgaccacgctacacaaaccatgcatgtcattgattcttatggatcactaactgttgggtatcacgtgctcaaggccggaactgtcaatcatttaattcagtttgcctcaaatgatatgcaaagcgagatgaaacattacagagttggc

SEQ ID NO: 8 - protein, 453 aa, Zucchini yellow mosaic virus
SQPEVQFFQGWRRMFDKFRPSLDHVCKVDHNNEECGELAAIFCQALFPVVKLSCQTCREKLSRVSFEEFKDSLNANFIIHKDEWDSFKEGSHYDNIFKLIKVATQATQNLKLSSEVMKLVQNHTSTHMKQIQDINKALMKGSLVTQDELDLALKQLLEMTQWFKNHMHLTGEEALKMFINKRSSKAMINPSLLCDNQLDKNEILFGEKEIHSKRLFKNFFEEVYQRRIYEVRSANFPNGTRKLAIGSLIVPLNLDRARTALLGESIEKKPLTSACVSQQNGNYIHSCCCVTMDDGTPMYSELKSPTKRHLVIGASGDPKYIDLPASEAERMYIAKEGYCYLNIFLAMLVNVNENEAKDFTKMIRDVLIPMLGQWPSLMDVATAAYILGVFHPETRCAELPRILVDHATQTMHVIDSYGSLTVGYHVLKAGTVNHLIQFASNDMQSEMKHYRVG

SEQ ID NO: 9 - DNA, 837 bp, Zucchini yellow mosaic virus
tcaggcactcagccaactgtggcagacactggagctacaaagaaagataaagaagatgacaaagggaaaaacaaggacgttacaggctccggctcaggtgagaaaacagtagcagctgtcacgaaggacaaggatgtgaatgctggttctcatgggaaaattgtgccgcgtctttcgaagatcacaaagaaaatgtcattgccacgcgtgaaaggaaatgtgatactcgatattgatcatttgctggaatataaaccggatcaaattgagttatataacacacgagcgtctcatcagcagttcgcctcttggttcaaccaggttaagacggaatatgatttgaacgagcaacagatgggagttgtaatgaatggtttcatggtttggtgcattgagaatggcacttcacccgacattaatggagtgtgggttatgatggacggaaatgagcaagttgagtatcccttgaaaccaatagttgaaaatgcaaagccaacgctgcggcaaataatgcatcatttttcagatgcagcggaggcatatatagagatgagaaatgcagaggcaccatacatgccgaggtatggtttgcttcgaaacctacgggataggagtttagcacgatatgcttttgatttctatgaagtcaattctaaaactcctgaaagagcccgcgaagctgttgcgcagatgaaagcagcagctcttagcaatgtttcttcaaggttgtttggccttgatggaaatgttgccaccactagcgaagacactgaacggcacactgcacgtgatgttaatagaaacatgcacaccttactaggtgtgaatacaatgcag

SEQ ID NO: 10 - protein, 279 aa, Zucchini yellow mosaic virus
SGTQPTVADTGATKKDKEDDKGKNKDVTGSGSGEKTVAAVTKDKDVNAGSHGKIVPRLSKITKKMSLPRVKGNVILDIDHLLEYKPDQIELYNTRASHQQFASWFNQVKTEYDLNEQQMGVVMNGFMVWCIENGTSPDINGVWVMMDGNEQVEYPLKPIVENAKPTLRQIMHHFSDAAEAYIEMRNAEAPYMPRYGLLRNLRDRSLARYAFDFYEVNSKTPERAREAVAQMKAAALSNVSSRLFGLDGNVATTSEDTERHTARDVNRNMHTLLGVNTMQ

SEQ ID NO: 11 - DNA, 561 bp, Homo sapiens
atgaccaacaagtgtctcctccaaattgctctcctgttgtgcttctccacgacagctctttccatgagctacaacttgcttggattcctacaaagaagcagcaattgtcagtgtcagaagctcctgtggcaattgaatgggaggcttgaatactgcctcaaggacaggaggaactttgacatccctgaggagattaagcagctgcagcagttccagaaggaggacgccgcagtgaccatctatgagatgctccagaacatctttgctattttcagacaagattcatcgagcactggctggaatgagactattgttgagaacctcctggctaatgtctatcatcagagaaaccatctgaagacagtcctggaagaaaaactggagaaagaagatttcaccaggggaaaacgcatgagcagtctgcacctgaaaagatattatgggaggattctgcattacctgaaggccaaggaggacagtcactgtgcctggaccatagtcagagtggaaatcctaaggaacttttacgtcattaacagacttacaggttacctccgaaac

SEQ ID NO: 12 - protein, 187 aa, Homo sapiens [sequence omitted]

SEQ ID NO: 13 - DNA, 498 bp, Homo sapiens
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccag

SEQ ID NO: 14 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 15 - DNA, 714 bp, Aequorea victoria
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaa

SEQ ID NO: 16 - DNA, 1809 bp, Arabidopsis arenosa
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaa

SEQ ID NO: 17 - DNA, 33 bp, Artificial
  synthetic oligonucleotide primer
atgctgcagaagactaatctttttctctttctc

SEQ ID NO: 18 - DNA, 42 bp, Artificial
  synthetic oligonucleotide primer
tgactgcagcattacagtgtcaagctcatcatgtttgtatag

SEQ ID NO: 19 - DNA, 33 bp, Artificial
  synthetic oligonucleotide primer
aactgcagtcaatgttacgtcctgtagaaaccc

SEQ ID NO: 20 - DNA, 31 bp, Artificial
  synthetic oligonucleotide primer
acgcgtcgacctttgtttgcctccctgctgc